(19) United States
(12) Patent Application Publication
(10) Pub. No.: US 2006/0134602 A1
(43) Pub. Date: Jun. 22, 2006

(54) Methods of identifying cytotoxic effects in quiescent cells

(76) Inventors: Bruce A. Littlefield, Andover, MA (US); Kathleen A. Salvato, Manchester, NH (US); Laura Rudolph-Owen, Medford, MA (US)

Correspondence Address: Patent Department;CHOATE, HALL & STEWART LLP, ATTN.: Brenda Herschbach Jarrell, Ph.D., 53 State Street, Exchange Place, Boston, MA, 02109, US

(21) Appl. No.: 11/035,930

(22) Filed: Jan. 13, 2005

Related U.S. Application Data
(60) Provisional application No. 60/536,196, filed on Jan. 13, 2004.

(51) Int. Cl. C12Q 1/00

(52) U.S. Cl. 435/4

(57) ABSTRACT

The present invention provides a system for measuring the cytotoxicity of a test compound against quiescent cells. The inventive methods and reagents may also be used to determine whether a compound, such as a chemotherapeutic agent, will have undesired toxicity toward normal tissues, which generally do not include a significant number of cells that are proliferating. Thus, the assay of the invention can be used to identify in vitro compounds that demonstrate a good therapeutic index, thus, accelerating the development of drugs that are clinically useful.

### PRIORITY INFORMATION

The present application claims priority to provisional application U.S. Ser. No. 60/536,196 filed Jan. 13, 2004 the entire contents of which are hereby incorporated by reference.

### BACKGROUND OF THE INVENTION

Chemotherapeutic agents typically are designed to target a particular population of abnormal (e.g., cancerous) or undesirable (e.g., parasitic) cells in preference to normal cells present in a mammal. Typically, an undesirable cell population divides more rapidly than normal cells and can be targeted with chemotherapeutic agents that are more cytotoxic to dividing cells than to non-dividing cells. Therefore, most currently available in vitro assays are designed to select compounds that are cytotoxic to dividing cells and give little information regarding their cytotoxicity to normal non-dividing or slowly dividing cells. In vitro assays which can quantify cytotoxic effects against non-proliferating cells have not been described. Therefore, toxicity of a chemotherapeutic agent to mammals is typically determined by expensive and time consuming in vivo animal studies and clinical trials. Unfortunately, cytotoxicity against normal non-dividing cells within the context of affected organs is the most common reason that compounds fail in the clinic. There remains a need for the development of an in vitro assay which measures cytotoxic effects of candidate chemotherapeutic agents against non-dividing cells so that compounds selected for in vivo testing can be restricted to those compounds that are most likely to have a good therapeutic index in vivo. Such an assay would accelerate the development of drugs that are clinically useful.

### SUMMARY OF THE INVENTION

The present invention provides a system for measuring the cytotoxicity of a test compound against quiescent non-dividing cells. In one aspect, the invention provides an assay. The inventive methods and reagents may also be used to determine whether a compound, such as a chemotherapeutic agent, will have undesired toxicity toward normal tissues, which generally do not include a significant number of cells that are proliferating. Thus, the assay of the invention will improve the selection for in vivo testing of compounds that demonstrate therapeutic activity in vitro.

In general, the assay involves contacting test compounds with a population of quiescent cells. In preferred embodiments, at least about 95% of the cells in the population are quiescent. The present inventors have found that growth to confluence is not always sufficient to achieve a sufficiently quiescent cell population in a culture. Therefore, in certain preferred embodiments, cells grown to confluence are further subjected to serum starvation conditions. The present inventors have found that this strategy can ensure that at least about 95% of cells, and preferably at least about 98%, 99%, 99.9% or 99.99% of the cells, in a population enter a quiescent non-dividing state.

In one embodiment, the assay involves growing plated cells to confluence in a tissue culture medium containing at least about 5% animal sera, preferably between about 5% and about 20% animal sera; optionally washing the cells one or more times; serum starving the confluent cells in a tissue culture medium that contains between about 0% and about 0.5% animal sera, thereby obtaining a quiescent cell culture; incubating the quiescent cell culture with the test compound for at least about six hours; and determining the viability of the cells, thereby determining the cytotoxicity of the test compound. As will be appreciated by those of ordinary skill in the art serum starvation may occur in different amounts of time for different cell types. In general, cells will be starved for at least about six hours; typically at least about one day and often less than about ten days. In many embodiments, cells are serum starved for about one to about three days.

In another aspect, the invention provides kits and reagents for measuring the cytotoxicity of a test compound towards quiescent cells. Preferred kits include a compound that inhibits cell proliferation and a cytotoxic compound. Inventive kits may also include a cell culture and/or one or more tissue culture medium, and/or one or more reagents for measuring the viability of cells.

### Definitions

The term “confluence,” as used herein, refers to a condition of a cell culture wherein the cells have grown a continuous layer over a cell culture matrix, such as a cell culture well. Typically, cell proliferation will slow down once confluence is reached because cells usually grow best when attached to a solid surface.

The term “cytotoxicity,” as used herein, refers to the ability of a compound to cause cell death or apoptosis induction. Cytotoxicity of compounds is typically compared by comparing the IC₅₀ of the compounds (i.e., the concentration of the compound that will kill half of the cells).

The term “quiescent,” as used herein, when referring to a single cell in a population indicates that the cell is in a Go state. When the term “quiescent” is used herein to refer to a cell culture, it indicates that at least about 95% of the cells are in a Gₒ state. In some embodiments, at least about 98%, 99%, 99.9% or 99.99% of the cells in a quiescent cell culture are in the Gₒ state.

### BRIEF DESCRIPTION OF THE DRAWING

The invention is described with reference to the several figures of the drawing, in which,

FIGS. 1 through 4 are graphs showing [³H]-thymidine uptake by IMR-90 human lung fibroblast cells that have been subjected to different conditions during a growth phase and a serum starvation phase.

FIG. 5 is a graph showing the cytotoxic effects of 2,4-dinitrophenol (DNP), carbonyl cyanide p-(trifluoromethoxy)phenyl hydrazone (carbonyl cyanide) and ouabain against quiescent IMR-90 human lung fibroblast cells.

FIGS. 6A through 6F are graphs showing the cytotoxic effects of 5-fluorouricil (5-FU), paclitaxel and vinblastine against quiescent IMR-90 human lung fibroblast cells. The cytotoxicity of carbonyl cyanide is shown as a positive control.

FIGS. 7A through 7F are graphs showing the cytotoxic effects of doxorubicin, SN-38 (an active metabolite of irinotecan (CPT-11)) and oxaliplatin against quiescent IMR-90 human lung fibroblast cells. The cytotoxicity of carbonyl cyanide is shown as a positive control.

FIGS. 8A through 8F are graphs showing the cytotoxic effects of actinomycin D, vincristine and cycloheximide against quiescent IMR-90 human lung fibroblast cells. The cytotoxicity of carbonyl cyanide is shown as a positive control.

FIGS. 9A through 9F are graphs showing the cytotoxic effects of etoposide, mitomycin C and gemcitabine against quiescent IMR-90 human lung fibroblast cells. The cytotoxicity of carbonyl cyanide is shown as a positive control.

### DETAILED DESCRIPTION OF CERTAIN PREFERRED EMBODIMENTS

The present invention provides a system for measuring the cytotoxicity of a test compound against quiescent cells. Many agents that are therapeutically effective in vitro are found to have unacceptable toxicity in vivo. This is particularly true of chemotherapeutic agents, which are supposed to preferentially kill proliferating cells while sparing normal non-proliferating cells. The inventive system allows researchers to better select therapeutically active compounds for in vivo testing because the assay measures the cytotoxicity of compounds against non-proliferating cells.

Cells that can be used in the assay of the invention include any non-malignant primary cell culture or cell line. Examples of cells that can be used include fibroblasts, myoblasts, mesenchymal cells, endothelial cells, chondrocytes, hepatocytes, Islet cells, nerve cells, muscle cells, hematopoietic cells, bone forming cells, stem cells, connective tissue stem cells, mesodermal stem cells, and epithelial cells. In one embodiment, the cells are IMR-90 human lung fibroblasts.

The type of tissue culture medium used in the method of the invention is not particularly restricted. Typically, the type of cell to be grown is taken into account when selecting a tissue culture medium. Examples of tissue culture media used to culture animal cells include, but are not limited to, Eagle's minimum essential medium, Eagle's minimum essential medium with Earle's salts, Eagle's minimum essential medium with Hank's salts, Eagle's minimum essential medium with L-glutamine and without salts and sodium bicarbonate, Dulbecco's modified Eagle's medium, Glasgow minimum essential medium, RPMI-1640 medium, hepatocyte medium, basal medium Eagle with Earle's salts, basal medium Earle with Hank's salts, basal medium Eagle without salts, Fischer's medium with L-glutamine and without sodium bicarbonate, Iscove's modified Dulbecco's medium with L-glutamine and 25 mM HEPES buffer and without sodium bicarbonate, Leibovitz-15 medium with L-glutamine and without antibiotics and sodium bicarbonate, Lactalbumin hydrolysate medium with Earle's salts and without sodium bicarbonate, Lactalbumin hydrolysate medium with Hank's salts and without sodium bicarbonate, LY medium without sodium bicarbonate, McCoy's 5a medium, Medium 199 base with L-glutamine and without salts and sodium bicarbonate, Medium 199 with Earle's salts, Medium 199 with Hank's salts, nutrient mixture F-10 (HAM), nutrient mixture F-12 (HAM) without L-glutamine and sodium bicarbonate, Puck's medium N-15 with L-glutamine without sodium bicarbonate, RPMI-1640, Scherer's maintenance medium without glycerol and sodium bicarbonate, SFRE medium 199-1, and Waymouth medium MB 752/1 with L-glutamine without sodium bicarbonate. Examples of tissue culture media used to culture plant cells include Anderson's Rhododendron medium, Gamborg's medium, Gerbera multiplication medium, Knudson solution-C medium, modified white root culture medium, Murashige and Skoog medium, Nitsch medium, Street medium, and White's medium. A person of ordinary skill in the art would be able to select the appropriate type of tissue culture medium to grow a particular cell type.

The type of animal sera used in the method of the invention is also not particularly restricted. Typical considerations taken into account when selecting an animal sera include the composition of the tissue culture medium, the type of cell to be grown and the culture system that is employed. Examples of animal sera include bovine serum (including fetal bovine serum), equine serum, porcine serum, human serum, chicken serum, rabbit serum, sheep serum, and goat serum. Combinations of two or more types of animal sera may also be employed. In one embodiment, the animal serum is fetal bovine serum. Typically, to facilitate growth of the cells to confluence, a tissue culture medium will include between about 5% and about 20% animal sera. In one embodiment, the cells are grown to confluence in a tissue culture medium that contains about 10% animal serum.

The time it takes to grow the cells to confluence is dependent on a number of factors including the type of cell, the composition of the tissue culture medium and the animal serum used during the growth phase, and the number of cells plated. In general, for most embodiments, growth for at least about six hours will be required to achieve confluence. Typically, it will take at least about one day, and often about three to about eight days (e.g., three to four days or seven to eight days for different cell types or culture conditions); usually less than about ten days, for cells plated on a tissue culture medium containing between about 5% and about 20% animal serum to grow to confluence. In one embodiment, between about 1.0×10² and about 5.0×10⁶ cells are plated per culture. When using a tissue culture plate the preferred number of cells may depend on the size of the well. For example, when using a 96-well plate, between about 1.0×10³ and about 1.0×10⁵ cells are preferably plated per well. A lower range is preferred for tissue culture plates with smaller wells (e.g., a 384-well plate) and a higher range is preferred for plates with larger wells (e.g., 6-well, 12-well or 48-well plates). A person of ordinary skill in the art would be able to determine when a cell culture has reached confluence.

After the cells are grown to confluence, the cell culture is preferably washed one or more times. Typically, the wash solution will be a tissue culture medium that contains either the same amount of animal serum as the serum starvation medium or less. In one embodiment, the cell culture is washed with a tissue culture medium that does not contain animal sera. Typically, the culture is washed from one to five times. Then the culture is incubated with a tissue culture medium that contains a low amount of animal sera or no animal sera to serum starve the cells. Typically, the tissue culture medium used during the serum starvation phase contains between 0% animal serum and about 0.5% animal serum. In a particular embodiment, the tissue culture medium in which the cells are serum starved contains about 0.1% animal serum.

The cells are serum starved for a time period necessary to achieve a quiescent cell culture having at least 95% of cells in the Gₒ state. Preferably, the cells are serum starved for a time period necessary to achieve a quiescent cell culture having at least about 98%, 99%, 99.9% or 99.99% of the cells in the Gₒ state. Typically, to achieve a quiescent cell culture, the cells in a cell culture are serum starved for at least about six hours. More commonly, cells are serum starved for at least about one day, often at least about two to about four days, and usually less than about ten days to obtain a quiescent cell culture. In another embodiment, the cells are serum starved for between about one and about three days.

The percentage of cells in a culture that are in the Gₒ state (i.e., non-proliferating) can be measured by any method known to those skilled in the art. For example, incorporation of [³H]-thymidine by the culture can be measured after the serum starvation step. Cells that are in the Gₒ state will not incorporate [³H]-thymidine since they are not proliferating. Incorporation of [³H]-thymidine can be measured at multiple time points during the serum starvation step to determine the time point wherein they are maximally quiescent (i.e., [³H]-thymidine incorporation will not decrease further with further serum starvation).

In addition, a positive control for a quiescent cell culture may be prepared by treating the serum starved cells with a compound that inhibits cell proliferation, such as aphidicolin. The positive control culture can be used to approximate the degree of quiescence of a culture after serum starvation.

Once the cell culture has been made quiescent using the method of the invention, it can be incubated with a test compound to determine the cytotoxicity of the test compound towards quiescent cells. The assay of the invention can be used to determine the cytotoxicity of any compound which mammals, especially humans, come in contact with by, for example, skin contact, inhalation, ingestion, injection, etc. For example, the assay of the invention can be used to determine the cytotoxicity of therapeutic agents, such as chemotherapeutic agents for cancer treatment, antimicrobial agents, antifungal agents, antiviral, psychotherapeutic agents, immunosuppressants, and the like; analgesic agents; pharmaceutical additives or carriers; diagnostic agents, such as fluorescent dyes; prophylactic products, such as vitamins, sunscreens, and weight loss products; personal care products and the constituents thereof, such as hair dyes, soaps, deodorants, toothpastes, and the like; food additives; products used in construction or home repair, such as paints, glues and adhesives; and industrial compounds, such as those used to make plastics or chemicals. In a preferred embodiment, the test compound is a chemotherapeutic agent that is a candidate drug for cancer treatment (e.g., DNA alkylating agents; agents that are incorporated into DNA and disrupts its function; agents which bind to microtubules and disrupts their synthesis; agents which stabilize microtubule formation and prevent their dissociation; agents which inhibit folic acid metabolism and thereby inhibit DNA synthesis; agents which inhibit angiogenesis; agents which inhibit enzymes involved in DNA synthesis; and chemosensitisers which decrease the ability of cancerous cells to develop drug resistance).

The concentration of a test compound that is incubated with the quiescent cell culture can be any concentration desired. However, typically the concentration is between about 0.001 nM and about 1 mM. In one embodiment, the test compound is dissolved in the serum medium starvation medium and added to the culture of quiescent cells. If the test compound is insoluble in the serum starvation medium, the test compound may be added to a small amount of a water soluble organic solvent, such as dimethyl sulfoxide (DMSO), methanol, ethanol, tetrahydrofuran (THF), and the like, and then added to the serum starvation medium. Typically, the organic solvent will comprise about 20% or less of the incubation solution containing the cytotoxic compound.

The test compound may be incubated with the quiescent cell culture for at least about six hours. In one embodiment, the test compound is incubated with the quiescent cell culture for between about six hours and about ten days. In another embodiment, the test compound is incubated with the quiescent cells culture for between about one day and about three days. Multiple incubation time points for a test compound may be taken. For example, a test compound may be incubated with multiple separate quiescent cell cultures for a different period of time in each culture. Then the viability of the cells in each culture can be determined. Alternatively, a test compound may be incubated with a single quiescent cell culture and cells from the culture may be sampled at multiple time points to determine their viability.

After the incubation period, the viability of the cells in the cell culture is determined by any direct or indirect method known to those skilled in the art. In one embodiment, the viability of cells in a culture is determined by determining the number of cells that are alive in the culture. For example, the number of cells that are alive in a culture that has been treated with a test compound can be determined by measuring the metabolic activity of the cells in the culture. For example, the amount of ATP produced by the culture or the mitochondrial reducing activity of the culture can be measured. ATP in a cell culture can be measured, for example, by reacting the ATP in the culture with luciferin in the presence of luciferase to produce light. The amount of light produced is a measure of the amount of ATP in the culture. Alternatively, viability of cells in a culture can be measured by determining the mitochondrial reducing activity of a cell culture by measuring NADH or NADPH in the cell culture. For example, NADH or NADPH can be measured by measuring the cleavage of tetrazolium salts to form formazan products. Typically, the results obtained for a quiescent culture that has been treated with a test compound are compared to a control quiescent cell culture that has been exposed to identical conditions except that it has not been exposed to the test compound. Data regarding ATP or formazan product production for the control quiescent cell culture may be obtained before, after, or concurrently with data for quiescent cultures treated with test compounds. The amount of decrease in ATP or formazan product production in the culture that have been treated with the test compound in comparison to the control culture is a measure of the cytotoxicity of the test compound.

In another embodiment, the number of viable cells in a culture can also be determined by determining the number of cells that maintain cell membrane integrity. For example, the number of viable cells in a quiescent cell culture that has be exposed to a test compound can be measured by determining the number of cells that exclude a dye, such as trypan blue, propidium iodide, or 7-aminoactinomycin D. The amount of increased dye absorption by the culture that have been treated with the test compound in comparison to a control culture is a measure of the cytotoxicity of the test compound. Data regarding dye absorption for the control quiescent cell culture may be obtained before, after, or concurrently with data for quiescent cultures treated with test compounds.

Alternatively, the viability of cells in a culture can be determined by determining the number of cells that are dead in a culture. For example, cell death in a culture can be determined by measuring the number of cells that have lost of cell membrane integrity, a hallmark of cell death. Typically, the number of cells that have lost membrane integrity in a culture that has been exposed to a test compound can be determined by measuring the leakage of one or more intracellular proteins (e.g., cytoplasmic enzymes or other proteins), such as lactate dehydrogenase, esterases or histones, from the cell and comparing it to the leakage of a similar cell culture that has not been incubated with the test compound. Alternatively, a quiescent cell culture can be incubated with Na₂(⁵¹Cr)O₄ before exposure to a test compound. Na₂(⁵¹Cr)O₄ will bind to most of the intracellular proteins. After the culture has been exposed to a test compound, the incubation solution is collected and the amount of gamma radiation in the solution is measured and compared to that of a control cell culture that has been similarly treated except that it has not been exposed to the test compound. The greater the radiation in the solution of the culture treated with the test compound compared to the control, the more cytotoxic the test compound. Data regarding the gamma radiation in a solution from the control quiescent cell culture may be obtained before, after or concurrently with data for quiescent cultures treated with test compounds.

In another embodiment, the viability of cells in a culture can be determined by determining the number of cells expressing apoptosis markers. For example, cells that have been incubated with a test compound that initiates apoptosis will express cell surface annexin V or activated caspase which can be measured and compared to a similar culture that has not been incubated with the test compound. Data regarding annexin V or activated caspase expression for the control quiescent cell culture may be obtained before, after or concurrently with data for quiescent cultures treated with test compounds.

The viability results of quiescent cell cultures that have been incubated with a test compound may also be compared with a positive control for cytotoxicity against quiescent cell cultures. For example, the results may be compared to the viability of a quiescent cell culture that has been incubated with a compound that is known to be cytotoxic. One example of compound having known cytotoxicity is carbonyl cyanide p-(trifluoromethoxy)phenyl-hydrazone. Positive control data may be obtained before, after or concurrently with data for quiescent cultures treated with test compounds.

In another aspect, the invention provides a kit for measuring the cytotoxicity of a test compound towards quiescent cells. Preferred kits may include a compound that inhibits cell proliferation; and a cytotoxic compound. The compound that inhibits cell proliferation can be used to treat a cell culture so that the treated culture can be used as a positive control standard to compare with other cell cultures to determine the extent of quiescence of the culture. In one embodiment, the compound that inhibits cell proliferation is aphidicolin. The compound that is cytotoxic can be incubated with a quiescent cell culture to provide a positive control for measuring the cytotoxicity of a test compound towards quiescent cells. In one embodiment, the cytotoxic compound is carbonyl cyanide p-(trifluoromethoxy)phenyl hydrazone.

Inventive kits can also include a culture of cells that may comprise any of the cells that can be used in the method of the invention. In addition, kits may include one or more of the tissue culture medium that can be used in the method of the invention. In one embodiment, the kit includes one tissue culture medium that includes between about 5% and about 20% animal sera and a second tissue culture medium that include between about 0% and about 0.5% animal sera. In another embodiment, a third tissue culture medium may also be included that includes no animal sera.

Kits of the invention may also include one or more reagents for measuring the viability of the cells in a cell culture. For example, a kit may include one or more reagents for measuring the metabolic activity of the cell culture. For example, luciferin and luciferase may be included in a kit to measure the ATP production of a cell culture; or a tetrazolium salt may be included in a kit to measure the mitochondrial reducing activity of the culture.

Alternatively, an inventive kit may include one or more reagents for measuring the number of cells that have intact cell membranes. For example, such a kit may include one or more dyes that are excluded from cells that have intact cell membranes but which enter into cells with less membrane integrity, such as dead or dying cells; or the kit may include one or more reagents that measure the release of one or more intracellular proteins (e.g., cytoplasmic enzymes or other proteins), such as lactate dehydrogenase, esterases or histones. Inventive kits may also include a radioactive compound, such as Na₂(⁵¹Cr)O₄ that will label one or more intracellular protein. The more radiolabeled proteins that are released from the cells of the culture, the fewer viable cells remain in the culture.

In another embodiment, the inventive kit may include one or more reagents for measuring the expression of an apoptosis marker. Since caspases are proteolytic enzymes that become activated during apoptosis, the kit may, for example, include a protein or peptide that when cleaved by a caspases will fluoresce. Similarly, the kit may include a protein or peptide that changes color when cleaved by a caspase. In addition, when a cell undergoes apoptosis, translocation of the membrane phospholipid phosphatidylserine from the inner to the outer side of the plasma membrane occurs. Annexin V binds to phosphatidylserine sites on the cell surface with a high affinity. Thus, a kit may include, for example, annexin V that has been conjugated to a dye or fluorochrome. Cells that are undergoing apoptosis are stained by binding the conjugated annexin V. It is to be understood that the invention is not limited to these specific reagents and that those skilled in the art will recognize other suitable reagents for measuring the expression of an apoptosis marker.

In another embodiment, an inventive kit may also include one or more tissue culture plates. For example, an inventive kit may include a 6-well, 12-well, 48-well, 96-well and/or 384-well tissue culture plate. The tissue culture plates may be made of a material that is translucent or transparent. If an assay that measures the production of light by the cell culture is used to measure the viability of cells in the cell culture, it can be advantageous to grow the cultures in a transparent or translucent tissue culture plate because the light produce by each culture may be measured without transfer from the tissue culture plate.

The invention is further illustrated by the following examples which are not intended to be limiting in any way.

### EXAMPLES

I. Preparation of Quiescent Cells-Seven Day Growth Phase and Four Day Serum Starvation Phase

Growth Conditions: Three 24 well plates (four row with 6 wells per row) were plated at 5×10⁴ cells/well, 1.0×10⁵ cells/well and 2.0×10⁵ cells/well in 1 mL of growth medium with IMR-90 human lung fibroblasts. The growth medium used was minimum essential medium (MEM) with Earle's salt and included 10% fetal bovine serum. The cells were incubated for seven days at 37° C. until they appeared confluent. The plate having the lowest seeding density (5×10⁴ cells/well) was discarded because the cells did not appear confluent.

Serum Starvation Conditions: The media was removed from the wells of the remaining two plates, and each well was washed twice with 1.5 mL of MEM with Earle's salt and no serum. 0.95 mL/well of MEM with Earle's salt media containing sodium pyruvate, non-essential amino acids and L-glutamine was added to each well. The media added to each row contained the following amounts of fetal bovine serum:

| | |
|---|---|
| Row 1: | 10% fetal bovine serum |
| Row 2: | 0.5% fetal bovine serum |
| Row 3: | 0.1% fetal bovine serum |
| Row 4: | no serum |

Fetal growth factor (10 ng/mL) was added to each well in columns 3 and 4. Aphidicolin (30 μM), a DNA polymerase inhibitor, was added to each well in columns 5 and 6. The wells in column 1 and 2 contained no growth factor and no aphidicolin. The cells were incubated at 37° C. for four days.

Quantitation of Quiescence: After incubation, quiescence was measured by measuring the [³H]-thymidine uptake by the cells. A separate stock solution of [³H]-thymidine was prepared for each row by adding 15 μL of 0.1 mCi/mL [³H]-thymidine to 135 mL of media that was used in that row. 10 μL of the appropriate stock solution was added to each well, and the cells were incubated for 2 hours at 37° C. A second series of stock solutions of 5 mM thymidine in each media was prepared. The media containing [³H]-thymidine was removed from each well and 1 mL of the appropriate second stock solution was added to each well. The cells were incubated for 1 hour at 37° C. The media was removed from each well and placed in separate 50 mL tubes which were kept on ice. 200 μL of a trypsin-EDTA solution was added to each well, and the cells were incubated for 15 to 20 minutes. The trypsin-EDTA solution was removed from each well and transferred to the respective 50 mL tube. Each well was washed with PBS twice and each wash was added to the respective 50 mL tube. The contents of each 50 mL tube was filtered over a pre-wet GF/F 25 mm Whatman filter. Each filter was washed twice with 10 mL of PBS, three times with 10 mL of water and once with 10 mL of 90% ethanol, then dried. The filters were transferred to scintillation vials and 4 mL of Liquiscint was added to each vial.

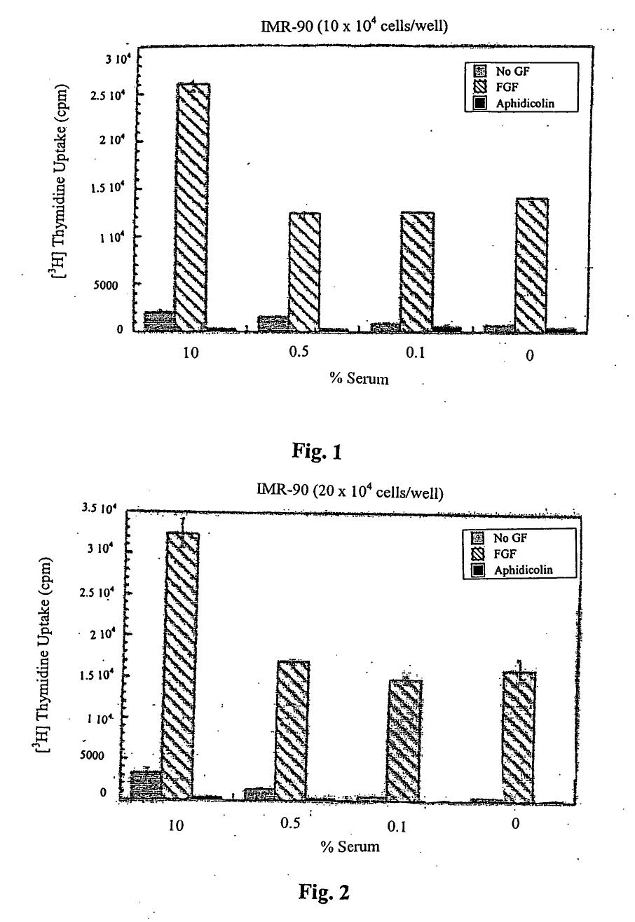

The results of the scintillation count are shown in FIGS. 1 and 2. A higher count indicates that more [³H]-thymidine has been incorporated into the DNA of the cells and indicates that the cells are less quiescent. Wells containing aphidicolin provide a standard for nearly totally quiescent cells. As can be seen by the graphs in FIGS. 1 and 2, cells incubated in media containing 0.1% fetal growth serum and no fibroblast growth factor (FGF) were nearly as quiescent as those containing aphidicolin demonstrating that this is the optimal condition for obtaining quiescent IMP-90 human lung fibroblast cells. The cells in columns 1 and 2 (wells containing no serum) were equally, or more, quiescent than those which were grown in 0.1% serum. However, cells grown in no serum appeared unhealthy and contained many cells that were not anchored to the walls of the well indicating that this was not the optimal condition for obtaining healthy quiescent cells.

II. Preparation of Quiescent Cells—Two Day Growth Phase and One Day Serum Starvation Phase

Growth Conditions: Two 96 well plates were plated at 1.6×10⁴ cells/well and 3.2×10⁴ cells/well, respectively, in 200 μL of growth medium (MEM with Earle's salt) with IMR-90 human lung fibroblasts. The cells were incubated at 37° C. for two days.

Cells did not appear confluent after two days growth.

Serum Starvation Conditions: The media was removed from the wells of the remaining two plates, and each well was washed twice with 200 μL of MEM with Earle's salt and no serum. 200 μL/well of MEM with Earle's salt media containing sodium pyruvate, non-essential amino acids, L-glutamine and 0.1% fetal bovine serum was added to each well and the cells were incubated for four day at 37° C. The media was removed from rows 2 through 8 and 200 μL/well of fresh MEM with Earle's salt media containing sodium pyruvate, non-essential amino acids, and L-glutamine was added to the wells. The media in each row contained the following amounts of fetal bovine serum and fetal growth factor:

| | |
|---|---|
| Rows 1 and 2: | 0.1% fetal bovine serum, no fetal growth factor |
| Rows 3 and 4: | 0.1% fetal bovine serum, 10 ng/mL fetal growth factor |
| Rows 5 and 6: | 10% fetal bovine serum, no fetal growth factor |
| Rows 7 and 8: | 10% fetal bovine serum, 10 ng/mL fetal growth factor |

30 μM of aphidicolin was added to the 12^{th} well in each row. The cells were incubated for one day at 37° C.

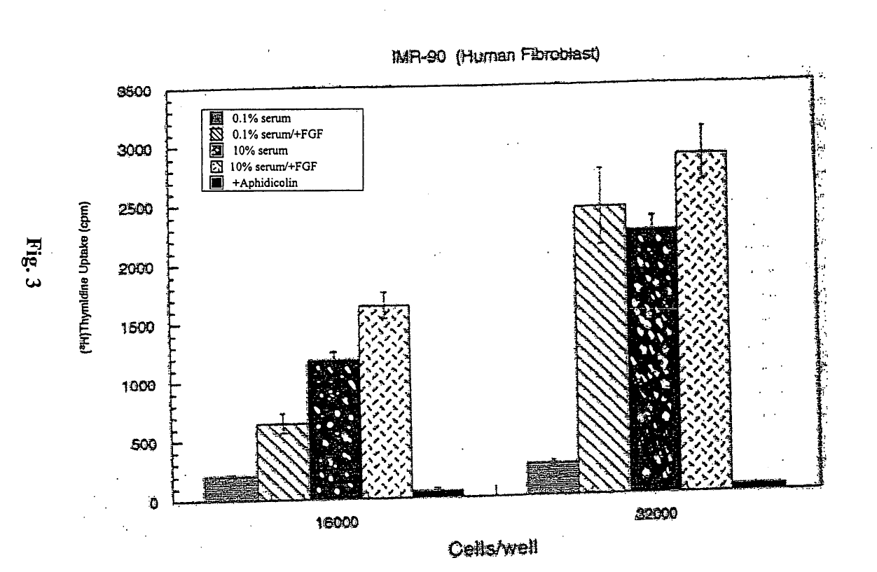

Quantitation of Quiescence: Quiescence of the cells was determined as in Example I. The results are shown in FIG. 3. Cells that were incubated in the serum starvation step with 0.1% fetal bovine serum and no fetal growth factor appeared nearly as quiescent as cells treated with aphidicolin. However, a longer growth phase that will allow cells to grow to confluence may further improve quiescence.

III. Preparation of Quiescent Cells—Four Day Growth Phase and Three Day Serum Starvation Phase

Growth Conditions: Two 96 well plates were plated at 1.6×10⁴ cells/well and 3.2×10⁴ cells/well, respectively, in 200 μL of growth medium (MEM with Earle's salt) with IMR-90 human lung fibroblasts. The cells were incubated at 37° C. for four days.

Serum Starvation Conditions: Serum starvation media was prepared and distributed among the 96 well plate as in Example II. As with Example II, aphidicolin was added to the 12^{th} well in each row.

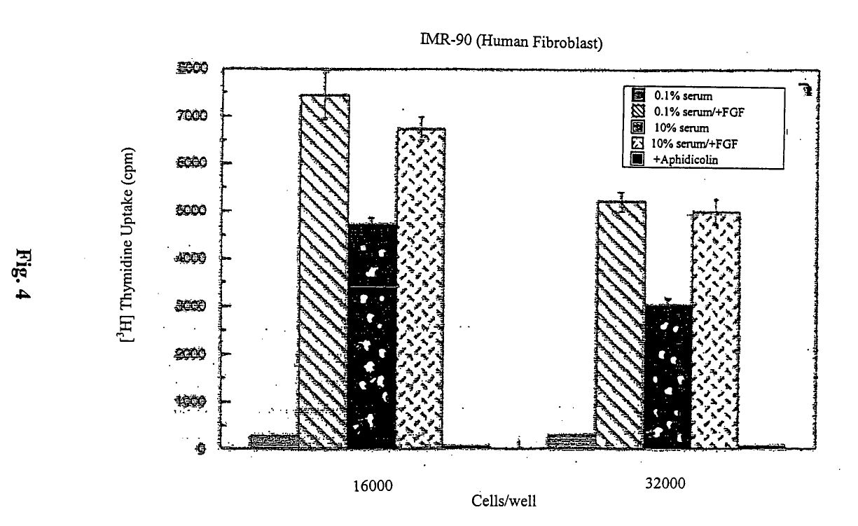

Quantitation of Quiescence: Quiescence was quantitated as in Example I. The results are shown in FIG. 4. Cells that were incubated in the serum starvation step with 0.1% fetal bovine serum and no fetal growth factor appeared nearly as quiescent as cells treated with aphidicolin.

IV. Selection of a Positive Control for Cytotoxicity Experiments

Preparation of Quiescent Cells: A 96 well plate was plated at 0.8×10⁴ cells/well in 200 μL of growth medium (MEM with Earle's salt) with IMR-90 human lung fibroblasts. The cells were incubated at 37° C. for six days.

The media was removed from the wells, and each well was washed twice with 200 μL of MEM with Earle's salt and no serum. 200 μL/well of MEM with Earle's salt media containing sodium pyruvate, non-essential amino acids, L-glutamine and 0.1% fetal bovine serum was added to each well, and the cells were incubated for one day at 37° C.

Selection of Positive Controls: 50 mM stock solutions of 2,4-dinitrophenol (DNP), carbonyl cyanide p-(trifluoromethoxy)phenyl hydrazone (carbonyl cyanide) and ouabain were prepared in dimethyl sulfoxide (DMSO). Aliquots of the 50 mM stock solution were diluted with DMSO to prepare stock solutions having concentrations of 0 μM, 0.03 μM, 0.1 μM, 0.3 μM, 1.0 μM, 3.0 μM, 10 μM, 30 μM, 100 μM, 300 μM, and 1000 μM.

The media was removed from each well and 90 μL/well of MEM with Earle's salt media containing sodium pyruvate, non-essential amino acids, L-glutamine and 0.1% fetal bovine serum was added to each well. 10 μL of DMSO with no compound was added to the wells in rows 1 and 2; 10 μL of each of the stock solution of carbonyl cyanide was added to two wells in rows 3 and 4; 10 μL of each of the stock solution of DNP stock solution was added to two wells in rows 5 and 6; and 10 μL of each of the stock solution of ouabain was added to two wells in rows 7 and 8 to obtain concentrations of the compounds in wells 2-11 of each row of 0 μM, 0.003 μM, 0.01 μM, 0.03 μM, 0.1 μM, 0.3 μM, 1.0 μM, 3.0 μM, 10.0 μM, 30.0 μM, and 100.0 μM, respectively. The first well in each row contained no cells. The cells were incubated at 37° C. for 24 hours.

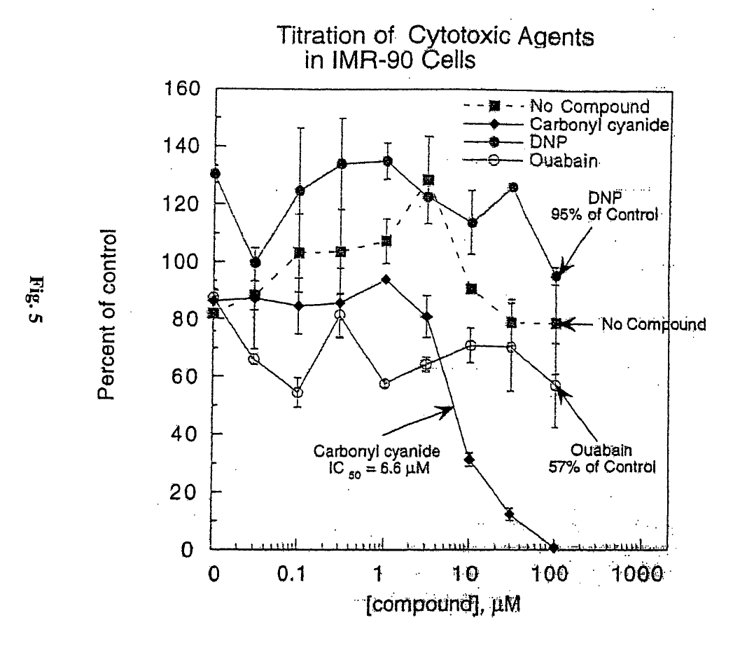

Quantitation of Cytotoxicity: A luciferase assay kit was used to quantitate the amount of ATP produced by cells in each well to measure cytotoxicity of the compounds. The assay utilizes the enzyme luciferase which catalyses a reaction of ATP and luciferin to form light. The light produced is measured to determine the amount of ATP produced by the cells in each well. The higher the cell mortality in each well, the less ATP, and consequently, the less light will be produced. FIG. 5 is a graph of the results for each of the compounds. Wells in rows one and two, which contained no compound, were controls. The results for each compound is reported as a percent of the control. As can be seen from FIG. 5, carbonyl cyanide was the only compound to completely kill the cells at the highest concentration tested and, thus, was selected as the best positive control for cytotoxicity.

V. Evaluation of Cytotoxicity of Test Compounds

Preparation of Quiescent Cells: A 96 well plate was plated at 0.8×10⁴ cells/well in 200 μL of growth medium (MEM with Earle's salt) with IMR-90 human lung fibroblasts. The cells were incubated at 37° C. for four days.

The media was removed from the wells, and each well was washed twice with 200 μL of MEM with Earle's salt and no serum. 90 μL/well of MEM with Earle's salt media containing sodium pyruvate, non-essential amino acids, L-glutamine and 0.1% fetal bovine serum was added to each well, and the cells were incubated for one day at 37° C.

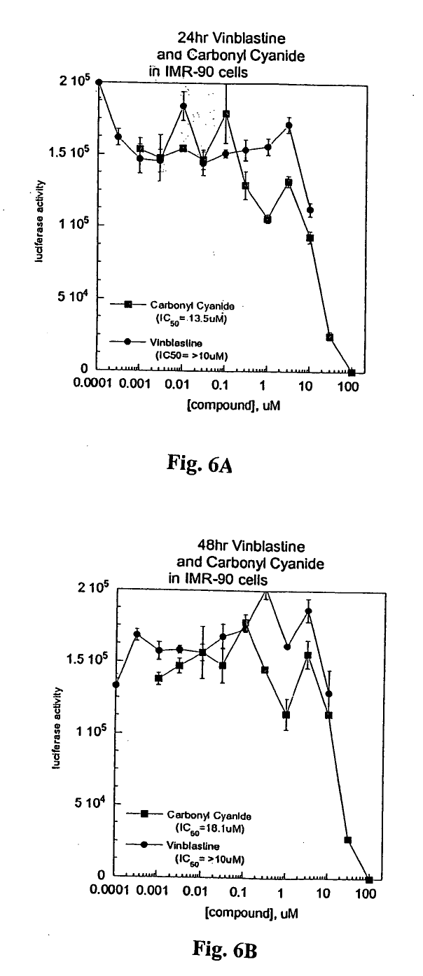

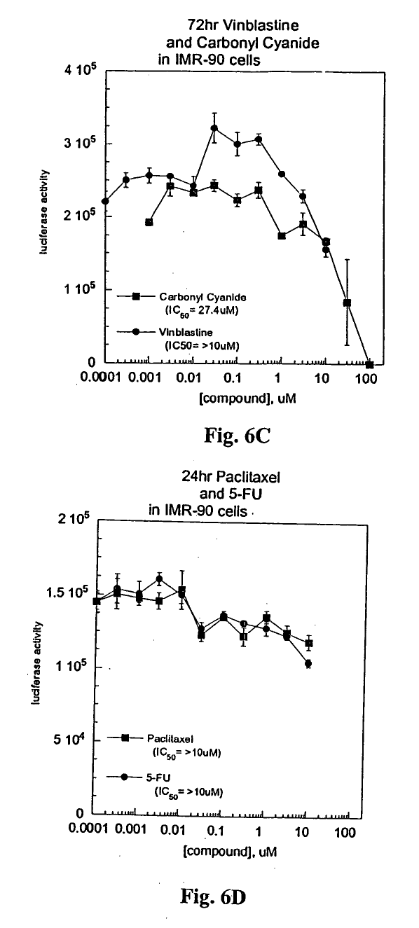

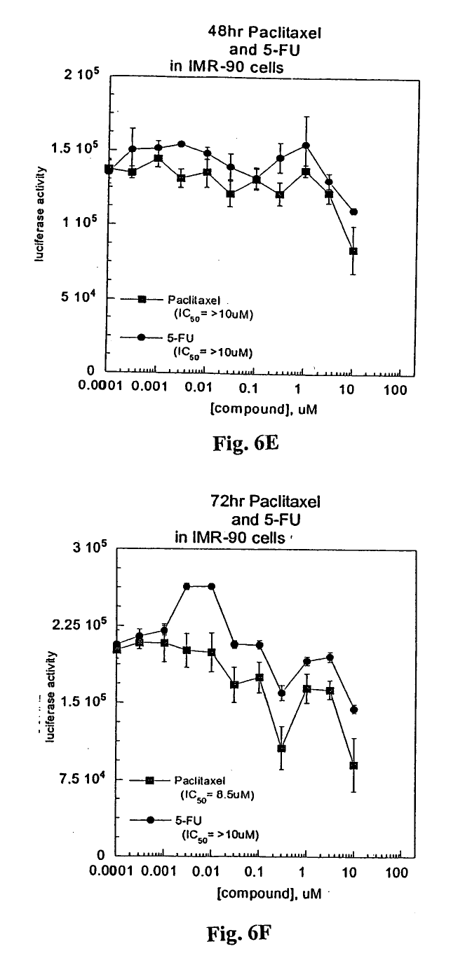

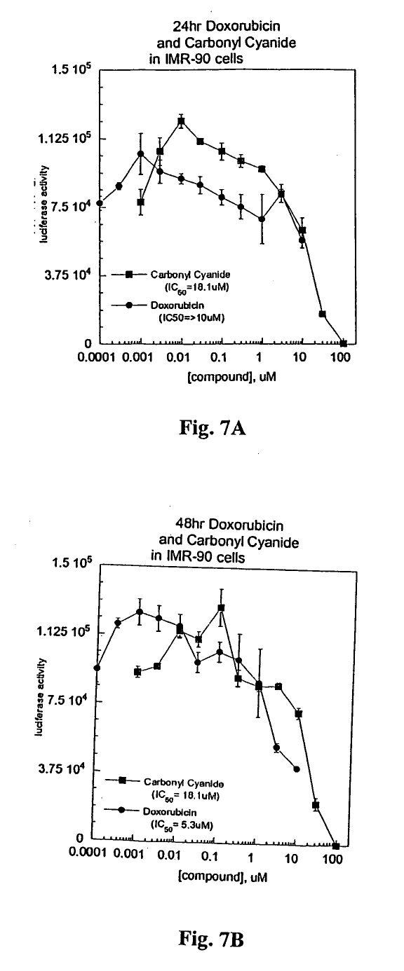

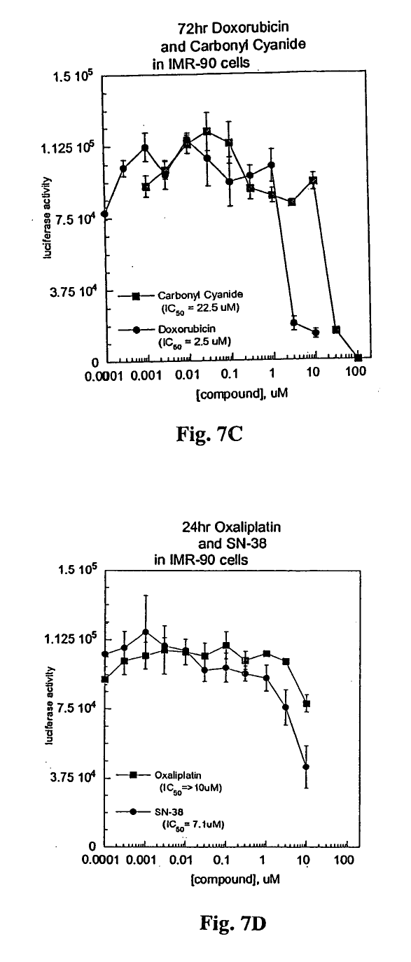

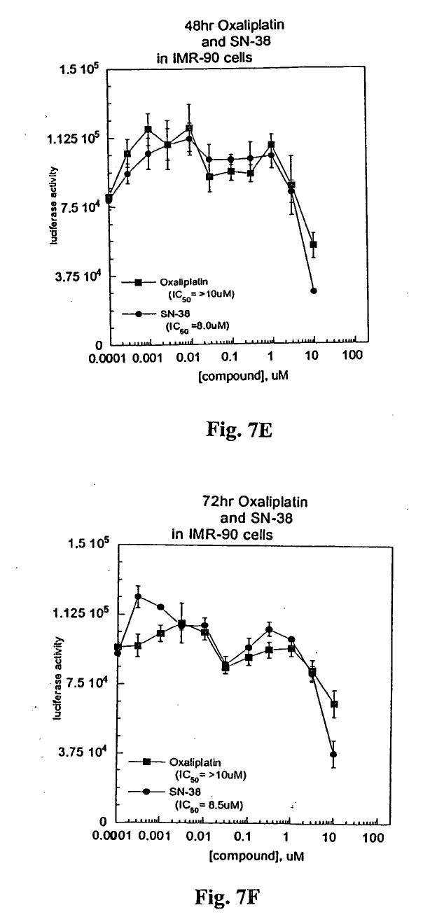

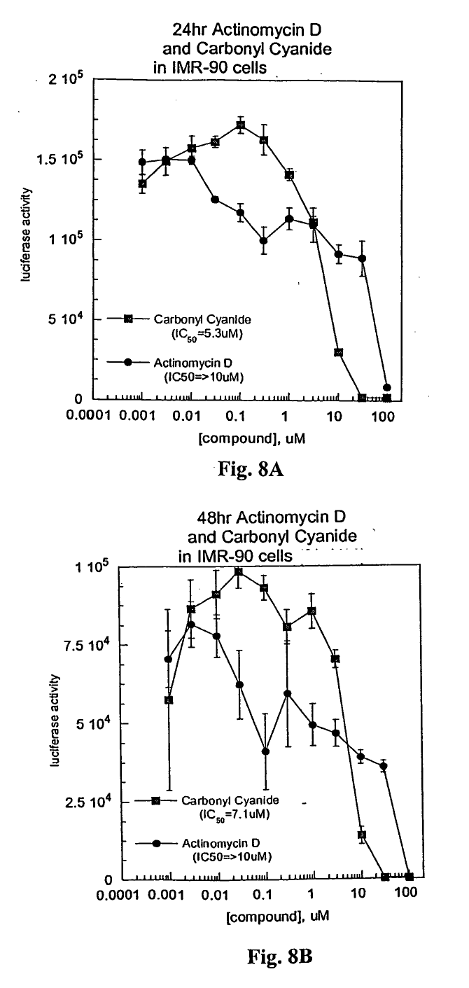

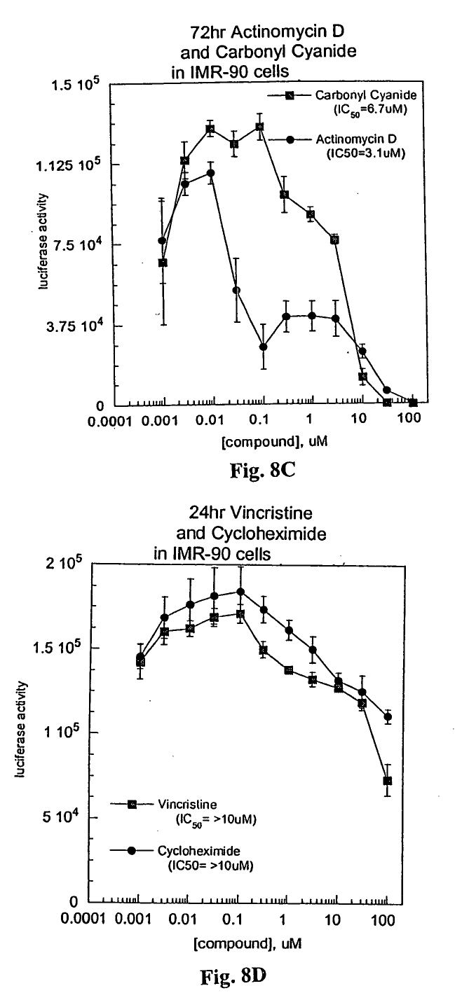

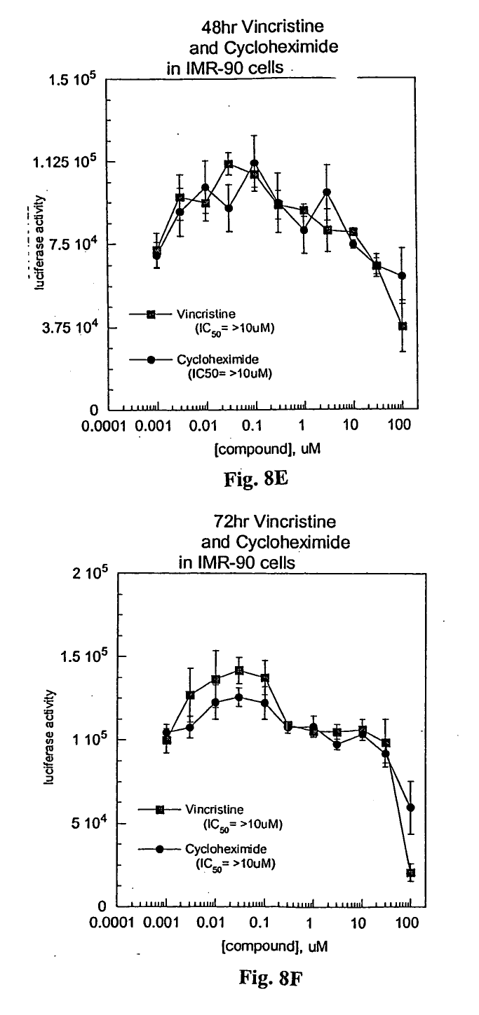

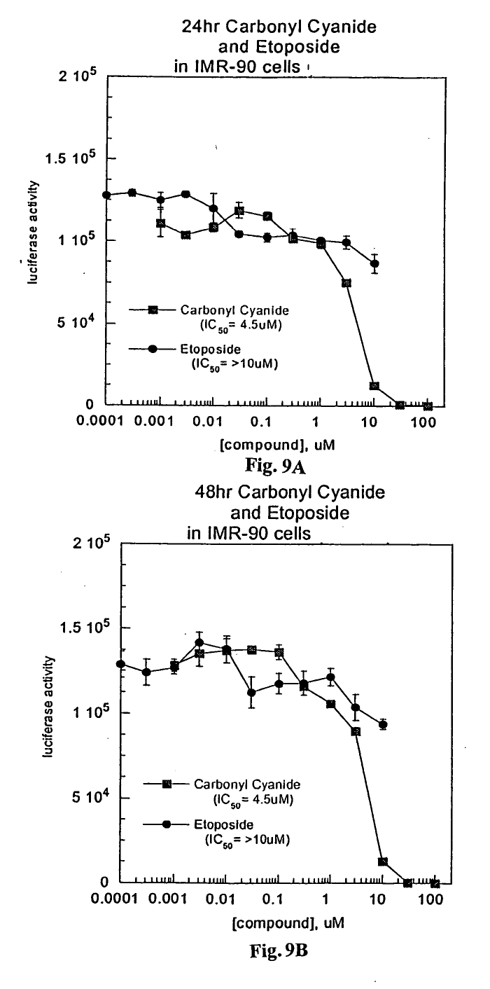

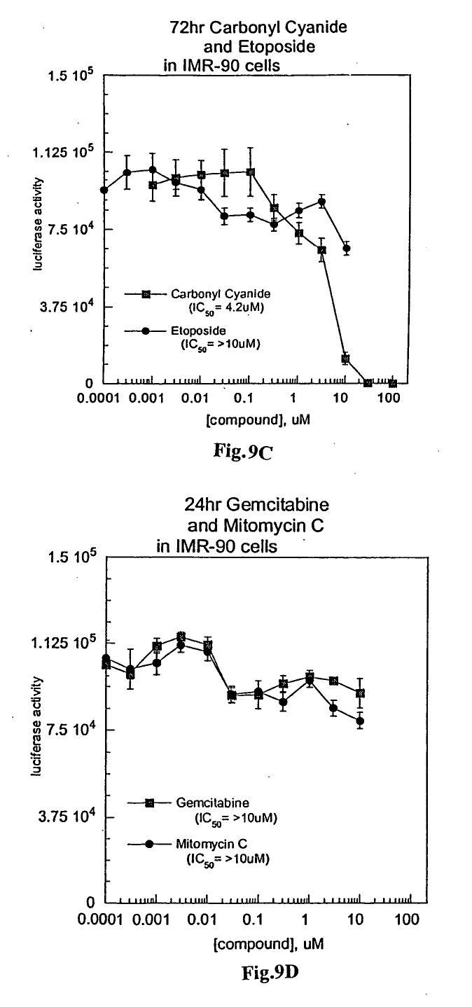

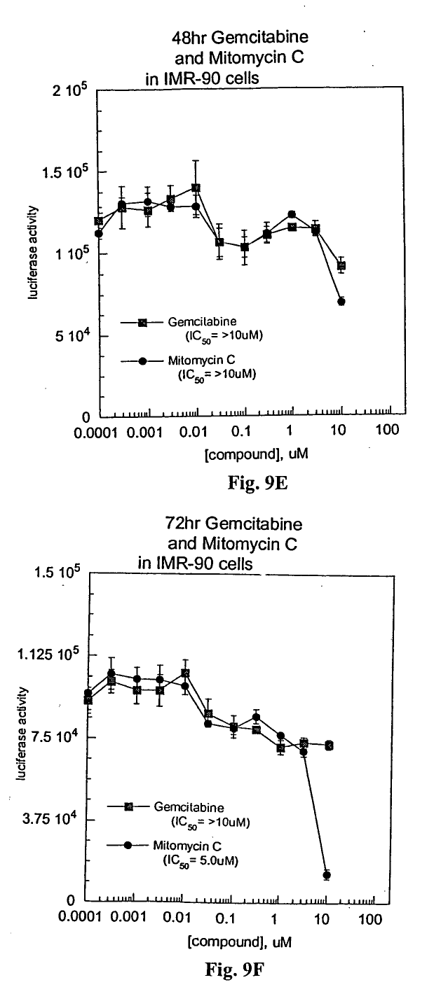

10 μL of carbonyl cyanide (a positive control for cytotoxicity) stock solutions were added to rows one and two to obtain concentrations of 0 μM, 0.003 μM, 0.01 μM, 0.03 μM, 0.1 μM, 0.3 μM, 1.0 μM, 3.0 μM, 10.0 μM, 30.0 μM, and 100.0 μM in wells 2 through 12. 10 μL of stock solution of test compounds were added to duplicate row to obtain concentrations of 0 μM, 0.003 μM, 0.01 μM, 0.03 μM, 0.1 μM, 0.3 μM, 1.0 μM, 3.0 μM, 10.0 μM, 30.0 μM, and 100.0 μM in wells 2 through 12. The first well in each row contained no cells. The compounds tested were clinical drugs vinblasine, 5-fluorouricil (5-FU), paclitaxel, doxorubicin, oxaliplatin, SN-38 (an active metabolite of irinotecan (CPT-11)), vincristine, etoposide, gemcitabine, and mitomycin C, as well as laboratory tools actinomycin D and cycloheximide. The cells were incubated with the test compounds for 24 hours, 48 hours, or 72 hours. Cytotoxicity results for vinblastine, paxlitaxel and 5-FU are shown in FIGS. 6A through 6F, cytotoxicity results for doxorubicin, oxaliplatin, and SN-38 are shown in FIGS. 7A through 7F, cytotoxicity results for actinomycin D, vincristine, and cycloheximide are shown in FIGS. 8A through 8F, and cytotoxicity results for etoposide, gemcitabine and mitomycin C are shown in FIGS. 9A through 9F. The IC50 values at 24 hours, 48 hours and 72 hours for the compounds tested against quiescent human lung fibroblasts are shown in Table I.

The cytotoxicity results indicate that clinical agents which act solely within proliferative phases of the cell cycle show good cytotoxicity windows between cytotoxic effects on quiescent fibroblasts and growth inhibitory effects against proliferating cancer cells. The mitotic blocker paclitaxel, for example, inhibits cancer cell growth at low nanomolar concentration, yet is not cytotoxic to quiescent fibroblasts until approximately 10 μM even after 72 hours. Agents such as doxorubicin, mitomycin C, and actinomycin D, that act via direct interactions with nucleic acids, show time-dependent increases in cytotoxicity against quiescent fibroblasts. Overall, results from the spectrum of compounds tested indicates that agents with mechanisms which are exclusive to the proliferative phases of the cell cycle do not, generally show cytotoxicity towards quiescent fibroblasts.

TABLE I
Summary of IC₅₀ data for compounds tested against IMR-90 human lung
fibroblasts.

| Compound | 24 hours | 48 hours | 72 hours |
|---|---|---|---|
| Paclitaxel (n = 2) | >10 μM | >10 μM | 8.5 μM/10 μM |
| Oxaliplatin | >10 μM | >10 μM | >10 μM |
| 5-FU (n = 2) | >10 μM | >10 μM | >10 μM |
| Vinblastine (n = 2) | >10 μM | >10 μM | >10 μM |
| Vincristine | >10 μM | >10 μM | >10 μM |
| SN-38 | 7.1 μM | 8.0 μM | 8.5 μM |
| Doxorubicin | >10 μM | 5.3 μM | 2.5 μM |
| Etoposide | >10 μM | >10 μM | >10 μM |
| Gemcitabine | >10 μM | >10 μM | >10 μM |
| Mitomycin C | >10 μM | >10 μM | 5.0 μM |
| Atinomycin D | >10 μM | >10 μM | 3.1 μM |
| cycloheximide | >10 μM | >10 μM | >10 μM |

Other embodiments of the invention will be apparent to those skilled in the art from a consideration of the specification or practice of the invention disclosed herein. It is intended that the specification and examples be considered as exemplary only, with the true scope and spirit of the invention being indicated by the following claims.

1. A method of determining the cytotoxicity of a test compound to quiescent cells, comprising the steps of:
a) growing plated cells to confluence in a tissue culture medium containing between about 5% and about 20% animal sera;
b) washing the cells one or more times;
c) serum starving the cells for at least about six hours in a tissue culture medium that contains between about 0% and about 0.5% animal sera, thereby obtaining a quiescent cell culture;
d) incubating the quiescent cell culture with the test compound for at least about six hours; and
e) determining the viability of the cells, thereby determining the cytotoxicity of the test compound.

2. The method of claim 1, wherein the animal sera is selected from the group consisting of bovine serum, equine serum, porcine serum, human serum, chicken serum, rabbit serum, sheep serum, goat serum, and combinations thereof.

3. The method of claim 2, wherein the animal sera is fetal bovine serum.

4. The method of claim 1, wherein the cells are selected from the group consisting of fibroblasts, myoblasts, endothelial cells, chondrocytes, hepatocytes, Islet cells, nerve cells, muscle cells, bone forming cells, stem cells, connective tissue stem cells, mesodermal stem cells, and epithelial cells.

5. The method of claim 4, wherein the cells are IMR-90 human lung fibroblast cells.

6. The method of claim 1, wherein the tissue culture medium in which the cells are grown to confluence is selected from the group consisting of Eagle's minimum essential medium, Eagle's minimum essential medium with Earle's salts, Dulbecco's modified Eagle's medium, Glasgow minimum essential medium, RPMI-1640 medium, and hepatocyte medium.

7. The method of claim 6, wherein the tissue culture medium in which the cells are grown to confluence contains about 10% animal serum.

8. The method of claim 6, wherein the tissue culture medium in which the cells are grown to confluence is Eagle's minimum essential medium with Earle's salts, and further comprises L-glutamine, sodium pyruvate and non-essential amino acids.

9. The method of claim 8, wherein the tissue culture medium in which the cells are grown to confluence contains about 10% fetal bovine serum.

10. The method of claim 1, wherein the medium in which the cells are serum starved is selected from the group consisting of Eagle's minimum essential medium, Eagle's minimum essential medium with Earle's salts, Dulbecco's modified Eagle's medium, Glasgow minimum essential medium, RPMI-1640 medium, and hepatocyte medium.

11. The method of claim 10, wherein the tissue culture medium in which the cells are serum starved contains about 0.1% animal serum.

12. The method of claim 10, wherein the medium in which the cells are serum starved is Eagle's minimum essential medium with Earle's salts, and further comprises L-glutamine, sodium pyruvate and non-essential amino acids.

13. The method of claim 12, wherein the tissue culture medium in which the cells are serum starved contains about 0.1% fetal bovine serum.

14. The method of claim 1, wherein the cells are serum starved for between about six hours and about ten days.

15. The method of claim 14, wherein the cells are serum starved for between about one and about three days.

16. The method of claim 1, wherein between about 1.0×10² and about 5.0×10⁶ cells are plated.

17. The method of claim 16, wherein the cells are grown for between about one day and about ten days to achieve confluence.

18. The method of claim 16, wherein the cells are grown for between about three and about eight days to achieve confluence.

19. The method of claim 1, wherein the cells are plated in a 6-well, a 12-well, a 48-well, a 96-well or a 384-well plate and between about 1.0×10² and about 5.0×10⁶ cells are plated per well.

20. The method of claim 1, wherein the cells are plated in a 96-well plate and between about 1.0×10³ and about 1.0×10⁵ cells are plated per well.

21. The method of claim 1, wherein the cells are incubated with between about 0.001 nM and about 1 mM of the test compound.

22. The method of claim 21, wherein the test compounds are incubated with the cells in the tissue culture medium used to serum starve the cells.

23. The method of claim 1, wherein the cells are incubated with the test compound for between about six hours and about ten days.

24. The method of claim 23, wherein the cells are incubated with the test compound for between about one day and about three days.

25. The method of claim 1, wherein the viability of the cells is determined by measuring the metabolic activity of the cells.

26. The method of claim 25, wherein the metabolic activity of the cells is measured by measuring ATP produced by the cells.

27. The method of claim 25, wherein the metabolic activity of the cells is measured by measuring mitochondrial reducing activity by measuring NADH or NADPH.

28. The method of claim 27, wherein NADH or NADPH is measured by tetrazolium salt cleavage to form formazan products.

29. The method of claim 1, wherein the viability of the cells is determined by measuring the membrane integrity of the cells.

30. The method of claim 29, wherein the membrane integrity of the cells is measured by measuring exclusion of dye from the cells.

31. The method of claim 30, wherein the dye is trypan blue, propidium iodide, or 7-aminoactinomycin D.

32. The method of claim 29, wherein the membrane integrity of the cells is measured by measuring release of an intracellular protein from the cells.

33. The method of claim 32, wherein the intracellular protein is lactate dehydrogenase.

34. The method of claim 32, wherein the intracellular protein is selected from the group consisting of esterases, histones and combinations thereof.

35. The method of claim 29, wherein the cells are radiolabeled prior to incubation with the test compound and the membrane integrity of the cells is measured by measuring release of radioactive material from the cells.

36. The method of claim 35, wherein the cells are radiolabeled with Na₂(⁵¹Cr)O₄.

37. The method of claim 1, wherein the viability of the cells is determined by detecting an apoptosis marker.

38. The method of claim 37, wherein the apoptosis marker is detected by measuring the activation of caspases.

39. The method of claim 37, wherein the apoptosis marker is detected by annexin V staining.

40. The method of claim 1, further comprising the step of comparing the viability of quiescent cells obtained in step c) to quiescent cells incubated with the test compound.

41. The method of claim 1, further comprising the step of comparing the viability of quiescent cells incubated with the test compound to quiescent cells incubated with a cytotoxic compound.

42. The method of claim 41, wherein the cytotoxic compound is carbonyl cyanide p-(trifluoromethoxy)phenyl-hydrazone.

43. A kit for measuring the cytotoxicity of a test compound towards quiescent cells, comprising:
a) a compound that inhibits cell proliferation; and
b) a cytotoxic compound.

44. The kit of claim 43, wherein the compound that inhibits cell proliferation is aphidicolin.

45. The kit of claim 43, wherein the cytotoxic compound is carbonyl cyanide p-(trifluoromethoxy)phenyl hydrazone.

46. The kit of claim 43, further comprising a culture of cells.

47. The kit of claim 46, wherein the cells are selected from the group consisting of fibroblasts, myoblasts, endothelial cells, chondrocytes, hepatocytes, Islet cells, nerve cells, muscle cells, bone forming cells, stem cells, connective tissue stem cells, mesodermal stem cells, and epithelial cells.

48. The kit of claim 47, wherein the cells are IMR-90 human lung fibroblast cells.

49. The kit of claim 43, further comprising one or more tissue culture media.

50. The kit of claim 49, wherein one tissue culture medium has between about 5% and about 20% animal sera; and a second tissue culture medium has between about 0% and about 0.5% animal sera.

51. The kit of claim 50, further comprising a third tissue culture medium that has no animal sera.

52. The kit of claim 51, wherein the animal sera is fetal bovine serum.

53. The kit of claim 51, wherein the tissue culture medium is selected from the group consisting of Eagle's minimum essential medium, Eagle's minimum essential medium with Earle's salts, Dulbecco's modified Eagle's medium, Glasgow minimum essential medium, RPMI 1640 medium, and hepatocyte medium.

54. The kit of claim 43, further comprising one or more reagents for testing the viability of cells in a tissue culture.

55. The kit of claim 54, wherein the one or more reagents for testing the viability of cells is one or more reagents that measures the amount of ATP in the cells.

56. The kit of claim 55, wherein the one or more reagents are luciferase and luciferin.

57. The kit of claim 54, wherein the reagent for testing the viability of cells is a tetrazolium salt.

58. The kit of claim 54, wherein the reagent for testing the viability of cells is a dye.

59. The kit of claim 58, wherein the dye is trypan blue, propidium iodide, or 7-aminoactinomycin D.

60. The kit of claim 54, wherein the reagent for testing the viability of cells is a reagent that measures release of an intracellular protein from the cells.

61. The kit of claim 60, wherein the intracellular protein is lactate dehydrogenase.

62. The kit of claim 60, wherein the intracellular protein is selected from the group consisting of esterases, histones and combinations thereof.

63. The kit of claim 54, wherein the reagent for testing the viability of cells is Na₂(⁵¹Cr)O₄.

64. The kit of claim 43, further comprising one or more reagents for detecting an apoptosis marker.

65. The kit of claim 64, wherein the reagent for detecting an apoptosis marker is a protein or peptide that fluoresces when cleaved by a caspase.

66. The kit of claim 64, wherein the reagent for detecting an apoptosis marker is a protein or peptide that changes color when cleaved by a caspase.

67. The kit of claim 64, wherein the reagent for detecting an apoptosis marker is an annexin V stain.

68. The kit of claim 43, further comprising a 6-well tissue culture plate, a 12-well tissue culture plate, a 48-well tissue culture plate, a 96-well tissue culture plate, a 384-well tissue culture plate, or combinations thereof.

69. The kit of claim 68, wherein the tissue culture plate is translucent or transparent.
